(19) United States
(12) Patent Application Publication
(10) Pub. No.: US 2015/0119664 A1
(43) Pub. Date: Apr. 30, 2015

(54) SYSTEMS AND METHODS FOR IDENTIFYING VENTILATED BREATHING

(71) Applicant: Covidien LP, Mansfield, MA (US)

(72) Inventors: Paul S. Addison, Edinburgh (GB); James Ochs, Seattle, WA (US); James N. Watson, Dunfermline (GB)

(21) Appl. No.: 14/523,080

(22) Filed: Oct. 24, 2014

Related U.S. Application Data
(60) Provisional application No. 61/896,581, filed on Oct. 28, 2013.

(51) Int. Cl. A61B 5/1455; A61B 5/00; A61B 5/08

(52) U.S. Cl. CPC A61B 5/14551; A61B 5/0816; A61B 5/7278; A61B 5/7275
USPC 600/323

(57) ABSTRACT

Provided are systems and methods for processing a physiological signal in order to detect whether a patient's breathing is being controlled by a ventilator. A signal, such as a photoplethysmograph (PPG) may be processed to determine one or more various metrics indicative of the consistency of the patient's respiration.

### CROSS REFERENCE TO RELATED APPLICATION

This application claims the benefit of U.S. Provisional Application No. 61/896,581, filed Oct. 28, 2013, which is hereby incorporated by reference herein in its entirety.

### FIELD OF THE DISCLOSURE

The present disclosure relates to physiological signal processing, and more particularly relates to identifying ventilated breathing in a patient.

### SUMMARY

In some embodiments, provided is a computer-implemented method that comprises receiving a photoplethysmograph (PPG) signal, generating, using processing circuitry, at least one signal indicative of respiration consistency based on the PPG signal, identifying, using the processing circuitry, a presence of ventilated breathing based on the at least one signal indicative of respiration consistency, and providing, using the processing circuitry, an indicator of ventilated breathing based on the identifying of the presence of ventilated breathing.

In some embodiments, provided is a system comprising an input that receives a photoplethysmograph (PPG) signal from a sensor, and processing circuitry configured for generating at least one signal indicative of respiration consistency based on the PPG signal, identifying a presence of ventilated breathing based on the at least one signal indicative of respiration consistency, and providing an indicator of ventilated breathing based on the identifying of the presence of ventilated breathing.

In some embodiments, provided is a non-transitory computer-readable medium having computer program instruction stored thereon for performing the method comprising receiving a photoplethysmograph (PPG) signal, generating at least one signal indicative of respiration consistency based on the PPG signal, identifying a presence of ventilated breathing based on the at least one signal indicative of respiration consistency, and providing an indicator of ventilated breathing based on the identifying of the presence of ventilated breathing.

### BRIEF DESCRIPTION OF THE FIGURES

The above and other features of the present disclosure, its nature and various advantages will be more apparent upon consideration of the following detailed description, taken in conjunction with the accompanying drawings in which:

FIG. 1 shows an illustrative patient monitoring system in accordance with some embodiments of the present disclosure;

FIG. 2 is a block diagram of the illustrative patient monitoring system of FIG. 1 coupled to a patient in accordance with some embodiments of the present disclosure;

FIG. 3 shows an illustrative PPG signal that is modulated by respiration in accordance with some embodiments of the present disclosure;

FIG. 4 shows a comparison of portions of the illustrative PPG signal of FIG. 3 in accordance with some embodiments of the present disclosure;

FIG. 5 shows illustrative steps for determining respiration information from a PPG signal in accordance with some embodiments of the present disclosure;

FIG. 6 shows an illustrative PPG signal, a first derivative of the PPG signal, and a second derivative of the PPG signal in accordance with some embodiments of the present disclosure;

FIG. 7 shows illustrative steps for identifying a ventilated patient in accordance with some embodiments of the present disclosure;

FIG. 8 shows an illustrative autocorrelation signal in accordance with some embodiments of the present disclosure;

FIG. 9 shows illustrative steps for identifying a ventilated patient in accordance with some embodiments of the present disclosure; and

FIG. 10 shows an illustrative simplified scalogram of a patient with ventilated breathing in accordance with some embodiments of the present disclosure.

### DETAILED DESCRIPTION OF THE FIGURES

The present disclosure is directed towards respiratory monitoring. In many cases, when a patient's respiration is being monitored while the patient is on a ventilator, such as a ventilator that provides positive airway pressure at a desired inhalation/exhalation rate, the processing of physiological signals can be improved if it is known that the patient's breathing is under a ventilator's control. Indeed, in some cases, due to high frequency components introduced to a physiological signal, such as a photoplethysmograph (PPG) signal, by a ventilator, an erroneous respiration rate is displayed by a respiration rate monitor.

The present disclosure provides embodiments for processing a physiological signal, such as a PPG signal, in order to identify the presence of ventilated breathing.

For purposes of clarity, the present disclosure is written in the context of the physiological signal being a PPG signal generated by a pulse oximetry system. It will be understood that any other suitable physiological signal or any other suitable system may be used in accordance with the teachings of the present disclosure.

An oximeter is a medical device that may determine the oxygen saturation of the blood. One common type of oximeter is a pulse oximeter, which may indirectly measure the oxygen saturation of a patient's blood (as opposed to measuring oxygen saturation directly by analyzing a blood sample taken from the patient). Pulse oximeters may be included in patient monitoring systems that measure and display various blood flow characteristics including, but not limited to, the oxygen saturation of hemoglobin in arterial blood. Such patient monitoring systems may also measure and display additional physiological parameters, such as a patient's pulse rate.

An oximeter may include a light sensor that is placed at a site on a patient, typically a fingertip, toe, forehead or earlobe, or in the case of a neonate, across a foot. The oximeter may use a light source to pass light through blood perfused tissue and photoelectrically sense the absorption of the light in the tissue. In addition, locations that are not typically understood to be optimal for pulse oximetry serve as suitable sensor locations for the monitoring processes described herein, including any location on the body that has a strong pulsatile arterial flow. For example, additional suitable sensor locations include, without limitation, the neck to monitor carotid artery pulsatile flow, the wrist to monitor radial artery pulsatile flow, the inside of a patient's thigh to monitor femoral artery pulsatile flow, the ankle to monitor tibial artery pulsatile flow, and around or in front of the ear. Suitable sensors for these locations may include sensors for sensing absorbed light based on detecting reflected light. In all suitable locations, for example, the oximeter may measure the intensity of light that is received at the light sensor as a function of time. The oximeter may also include sensors at multiple locations. A signal representing light intensity versus time or a mathematical manipulation of this signal (e.g., a scaled version thereof, a log taken thereof, a scaled version of a log taken thereof, etc.) may be referred to as the photoplethysmograph (PPG) signal. In addition, the term “PPG signal,” as used herein, may also refer to an absorption signal (i.e., representing the amount of light absorbed by the tissue) or any suitable mathematical manipulation thereof. The light intensity or the amount of light absorbed may then be used to calculate any of a number of physiological parameters, including an amount of a blood constituent (e.g., oxyhemoglobin) being measured as well as a pulse rate and when each individual pulse occurs.

In some applications, the light passed through the tissue is selected to be of one or more wavelengths that are absorbed by the blood in an amount representative of the amount of the blood constituent present in the blood. The amount of light passed through the tissue varies in accordance with the changing amount of blood constituent in the tissue and the related light absorption. Red and infrared (IR) wavelengths may be used because it has been observed that highly oxygenated blood will absorb relatively less Red light and more IR light than blood with a lower oxygen saturation. By comparing the intensities of two wavelengths at different points in the pulse cycle, it is possible to estimate the blood oxygen saturation of hemoglobin in arterial blood.

When the measured blood parameter is the oxygen saturation of hemoglobin, a convenient starting point assumes a saturation calculation based at least in part on Lambert-Beer's law. The following notation will be used herein:

I(λ, t)=I_{O}(λ)exp(−(sβ_{O}(λ)+(1−s)βᵣ(λ))l(t)) (1)

where:
λ=wavelength;
t=time;
I=intensity of light detected;
I₀=intensity of light transmitted;
S=oxygen saturation;
β₀, βᵣ=empirically derived absorption coefficients; and
l(t)=a combination of concentration and path length from emitter to detector as a function of time.

The traditional approach measures light absorption at two wavelengths (e.g., Red and IR), and then calculates saturation by solving for the “ratio of ratios” as follows.
1. The natural logarithm of Eq. 1 is taken (“log” will be used to represent the natural logarithm) for IR and Red to yield

log I=log Iₒ−(sβ_{O}+(1−s)βᵣ)l. (2)
2. Eq. 2 is then differentiated with respect to time to yield

$$\frac{d log I}{d t} = - ( s {β}_{o} + ( 1 - s ) {β}_{r} ) \frac{d l}{d t} . \qquad ( 3 )$$

3. Eq. 3, evaluated at the Red wavelength λ_{R}, is divided by Eq. 3 evaluated at the IR wavelength λ_{IR} in accordance with

$$\frac{d log I ( {λ}_{R} ) / d t}{d log I ( {λ}_{IR} ) / d t} = \frac{s {β}_{o} ( {λ}_{R} ) + ( 1 - s ) {β}_{r} ( {λ}_{R} )}{s {β}_{o} ( {λ}_{IR} ) + ( 1 - s ) {β}_{r} ( {λ}_{IR} )} . \qquad ( 4 )$$

4. Solving for S yields

$$s = \frac{\frac{d log I ( {λ}_{IR} )}{d t} {β}_{r} ( {λ}_{R} ) - \frac{d log I ( {λ}_{R} )}{d t} {β}_{r} ( {λ}_{IR} )}{\frac{d log I ( {λ}_{R} )}{d t} ( \begin{matrix}{β}_{o} ( {λ}_{IR} ) - \\ {β}_{r} ( {λ}_{IR} )\end{matrix} ) - \frac{d log I ( {λ}_{IR} )}{d t} ( \begin{matrix}{β}_{o} ( {λ}_{R} ) - \\ {β}_{r} ( {λ}_{R} )\end{matrix} )} . \qquad ( 5 )$$

5. Note that, in discrete time, the following approximation can be made:

$$\frac{d log I ( λ , t )}{d t} ≃ log I ( λ , {t}_{2} ) - log ( λ , {t}_{1} ) . \qquad ( 6 )$$

6. Rewriting Eq. 6 by observing that log A−log B=log(A/B) yields

$$\frac{d log I ( λ , t )}{d t} ≃ log ( \frac{I ( {t}_{2} , λ )}{I ( {t}_{1} , λ )} ) . \qquad ( 7 )$$

7. Thus, Eq. 4 can be expressed as

$$\frac{\frac{d log I ( {λ}_{R} )}{d t}}{\frac{d log I ( {λ}_{IR} )}{d t}} ≃ \frac{log ( \frac{I ( {t}_{1} , {λ}_{R} )}{I ( {t}_{2} , {λ}_{R} )} )}{log ( \frac{I ( {t}_{1} , {λ}_{IR} )}{I ( {t}_{2} , {λ}_{IR} )} )} = R , \qquad ( 8 )$$

where R represents the “ratio of ratios.”
8. Solving Eq. 4 for S using the relationship of Eq. 5 yields

$$s = \frac{{β}_{r} ( {λ}_{R} ) - R {β}_{r} ( {λ}_{IR} )}{R ( {β}_{o} ( {λ}_{IR} ) - {β}_{r} ( {λ}_{IR} ) ) - {β}_{o} ( {λ}_{R} ) + {β}_{r} ( {λ}_{R} )} . \qquad ( 9 )$$

9. From Eq. 8, R can be calculated using two points (e.g., PPG maximum and minimum), or a family of points. One method applies a family of points to a modified version of Eq. 8. Using the relationship

$$\frac{d log I}{d t} = \frac{d I / d t}{I} , \qquad ( 10 )$$

Eq. 8 becomes

$$\frac{\frac{d log I ( {λ}_{R} )}{d t}}{\frac{d log I ( {λ}_{IR} )}{d t}} ≃ \frac{\frac{I ( {t}_{2} , {λ}_{R} ) - I ( {t}_{1} , {λ}_{R} )}{I ( {t}_{1} , {λ}_{R} )}}{\frac{I ( {t}_{2} , {λ}_{IR} ) - I ( {t}_{1} , {λ}_{IR} )}{I ( {t}_{1} , {λ}_{IR} )}} = \frac{[ I ( {t}_{2} , {λ}_{R} ) - I ( {t}_{1} , {λ}_{R} ) ] I ( {t}_{1} , {λ}_{IR} )}{[ I ( {t}_{2} , {λ}_{IR} ) - I ( {t}_{1} , {λ}_{IR} ) ] I ( {t}_{1} , {λ}_{R} )} = R , \qquad ( 11 )$$

which defines a cluster of points whose slope of y versus X will give R when

x=[I(t₂, λ_{IR})−I(t₁, λ_{IR})]I(t₁, λ_{R}), (12)

and

y=[I(t₂, λ_{R})−I(t₁, λ_{R})]I(t₁, λ_{IR}). (13)

Once R is determined or estimated, for example, using the techniques described above, the blood oxygen saturation can be determined or estimated using any suitable technique for relating a blood oxygen saturation value to R. For example, blood oxygen saturation can be determined from empirical data that may be indexed by values of R, and/or it may be determined from curve fitting and/or other interpolative techniques.

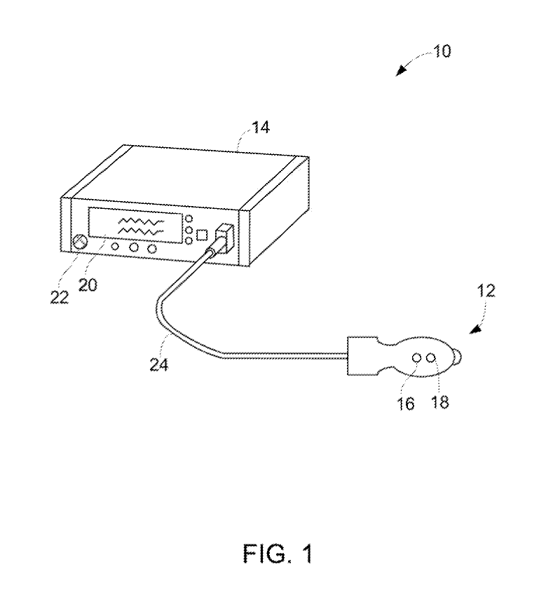

FIG. 1 is a perspective view of an embodiment of a patient monitoring system 10. System 10 may include sensor unit 12 and monitor 14. In some embodiments, sensor unit 12 may be part of an oximeter. Sensor unit 12 may include an emitter 16 for emitting light at one or more wavelengths into a patient's tissue. A detector 18 may also be provided in sensor unit 12 for detecting the light originally from emitter 16 that emanates from the patient's tissue after passing through the tissue. Any suitable physical configuration of emitter 16 and detector 18 may be used. In an embodiment, sensor unit 12 may include multiple emitters and/or detectors, which may be spaced apart. System 10 may also include one or more additional sensor units (not shown) that may take the form of any of the embodiments described herein with reference to sensor unit 12. An additional sensor unit may be the same type of sensor unit as sensor unit 12, or a different sensor unit type than sensor unit 12. Multiple sensor units may be capable of being positioned at two different locations on a subject's body; for example, a first sensor unit may be positioned on a patient's forehead, while a second sensor unit may be positioned at a patient's fingertip.

Sensor units may each detect any signal that carries information about a patient's physiological state, such as an electrocardiograph signal, arterial line measurements, or the pulsatile force exerted on the walls of an artery using, for example, oscillometric methods with a piezoelectric transducer. According to some embodiments, system 10 may include two or more sensors forming a sensor array in lieu of either or both of the sensor units. Each of the sensors of a sensor array may be a complementary metal oxide semiconductor (CMOS) sensor. Alternatively, each sensor of an array may be charged coupled device (CCD) sensor. In some embodiments, a sensor array may be made up of a combination of CMOS and CCD sensors. The CCD sensor may comprise a photoactive region and a transmission region for receiving and transmitting data whereas the CMOS sensor may be made up of an integrated circuit having an array of pixel sensors. Each pixel may have a photodetector and an active amplifier. It will be understood that any type of sensor, including any type of physiological sensor, may be used in one or more sensor units in accordance with the systems and techniques disclosed herein. It is understood that any number of sensors measuring any number of physiological signals may be used to determine physiological information in accordance with the techniques described herein.

In some embodiments, emitter 16 and detector 18 may be on opposite sides of a digit such as a finger or toe, in which case the light that is emanating from the tissue has passed completely through the digit. In some embodiments, emitter 16 and detector 18 may be arranged so that light from emitter 16 penetrates the tissue and is reflected by the tissue into detector 18, such as in a sensor designed to obtain pulse oximetry data from a patient's forehead.

In some embodiments, sensor unit 12 may be connected to and draw its power from monitor 14 as shown. In another embodiment, the sensor may be wirelessly connected to monitor 14 and include its own battery or similar power supply (not shown). Monitor 14 may be configured to calculate physiological parameters (e.g., pulse rate, blood oxygen saturation (e.g., SpO₂), and respiration information) based at least in part on data relating to light emission and detection received from one or more sensor units such as sensor unit 12 and an additional sensor (not shown). In some embodiments, the calculations may be performed on the sensor units or an intermediate device and the result of the calculations may be passed to monitor 14. Further, monitor 14 may include a display 20 configured to display the physiological parameters or other information about the system. In the embodiment shown, monitor 14 may also include a speaker 22 to provide an audible sound that may be used in various other embodiments, such as for example, sounding an audible alarm in the event that a patient's physiological parameters are not within a predefined normal range. In some embodiments, the system 10 includes a stand-alone monitor in communication with the monitor 14 via a cable or a wireless network link.

In some embodiments, sensor unit 12 may be communicatively coupled to monitor 14 via a cable 24. In some embodiments, a wireless transmission device (not shown) or the like may be used instead of or in addition to cable 24. Monitor 14 may include a sensor interface configured to receive physiological signals from sensor unit 12, provide signals and power to sensor unit 12, or otherwise communicate with sensor unit 12. The sensor interface may include any suitable hardware, software, or both, which may allow communication between monitor 14 and sensor unit 12.

As is described herein, monitor 14 may generate a PPG signal based on the signal received from sensor unit 12. The PPG signal may consist of data points that represent a pulsatile waveform. The pulsatile waveform may be modulated based on the respiration of a patient. Respiratory modulations may include baseline modulations, amplitude modulations, frequency modulations, respiratory sinus arrhythmia, any other suitable modulations, or any combination thereof. Respiratory modulations may exhibit different phases, amplitudes, or both, within a PPG signal and may contribute to complex behavior (e.g., changes) of the PPG signal. For example, the amplitude of the pulsatile waveform may be modulated based on respiration (amplitude modulation), the frequency of the pulsatile waveform may be modulated based on respiration (frequency modulation), and a signal baseline for the pulsatile waveform may be modulated based on respiration (baseline modulation). Monitor 14 may analyze the PPG signal (e.g., by generating respiration morphology signals from the PPG signal, generating a combined autocorrelation sequence based on the respiration morphology signals, and calculating respiration information from the combined autocorrelation sequence) to determine respiration information based on one or more of these modulations of the PPG signal.

As is described herein, respiration information may be determined from the PPG signal by monitor 14. However, it will be understood that the PPG signal could be transmitted to any suitable device for the determination of respiration information, such as a local computer, a remote computer, a nurse station, mobile devices, tablet computers, or any other device capable of sending and receiving data and performing processing operations. Information may be transmitted from monitor 14 in any suitable manner, including wireless (e.g., WiFi, Bluetooth, etc.), wired (e.g., USB, Ethernet, etc.), or application-specific connections. The receiving device may determine respiration information as described herein.

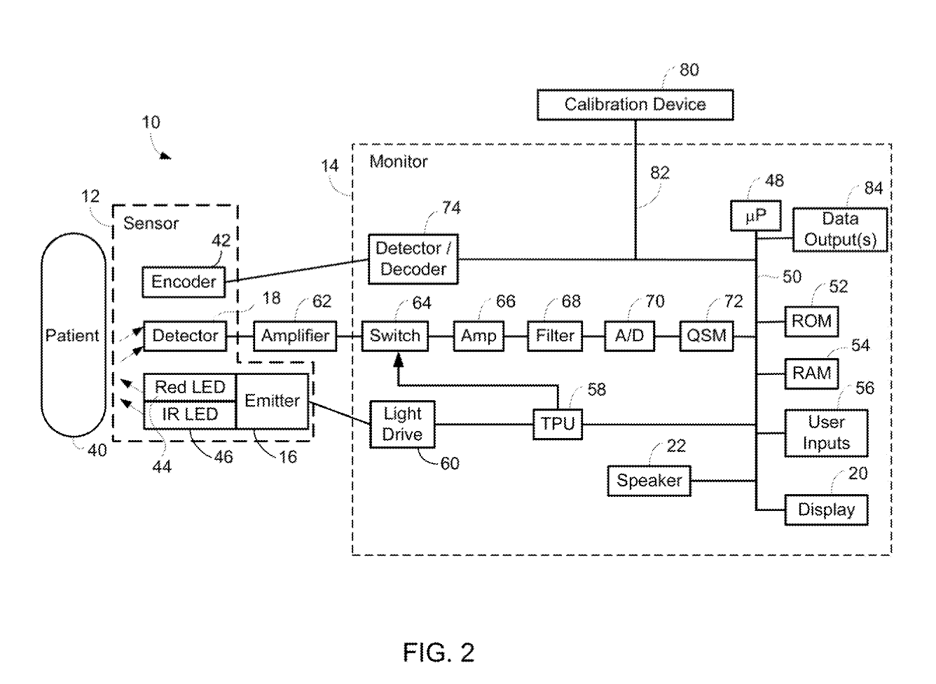

FIG. 2 is a block diagram of a patient monitoring system, such as patient monitoring system 10 of FIG. 1, which may be coupled to a patient 40 in accordance with an embodiment. Certain illustrative components of sensor unit 12 and monitor 14 are illustrated in FIG. 2.

Sensor unit 12 may include emitter 16, detector 18, and encoder 42. In the embodiment shown, emitter 16 may be configured to emit at least two wavelengths of light (e.g., Red and IR) into a patient's tissue 40. Hence, emitter 16 may include a Red light emitting light source such as Red light emitting diode (LED) 44 and an IR light emitting light source such as IR LED 46 for emitting light into the patient's tissue 40 at the wavelengths used to calculate the patient's physiological parameters. In some embodiments, the Red wavelength may be between about 600 nm and about 700 nm, and the IR wavelength may be between about 800 nm and about 1000 nm. In embodiments where a sensor array is used in place of a single sensor, each sensor may be configured to emit a single wavelength. For example, a first sensor may emit only a Red light while a second sensor may emit only an IR light. In a further example, the wavelengths of light used may be selected based on the specific location of the sensor.

It will be understood that, as used herein, the term “light” may refer to energy produced by radiation sources and may include one or more of radio, microwave, millimeter wave, infrared, visible, ultraviolet, gamma ray or X-ray electromagnetic radiation. As used herein, light may also include electromagnetic radiation having any wavelength within the radio, microwave, infrared, visible, ultraviolet, or X-ray spectra, and that any suitable wavelength of electromagnetic radiation may be appropriate for use with the present techniques. Detector 18 may be chosen to be specifically sensitive to the chosen targeted energy spectrum of the emitter 16.

In some embodiments, detector 18 may be configured to detect the intensity of light at the Red and IR wavelengths. Alternatively, each sensor in the array may be configured to detect an intensity of a single wavelength. In operation, light may enter detector 18 after passing through the patient's tissue 40. Detector 18 may convert the intensity of the received light into an electrical signal. The light intensity is directly related to the absorbance and/or reflectance of light in the tissue 40. That is, when more light at a certain wavelength is absorbed or reflected, less light of that wavelength is received from the tissue by the detector 18. After converting the received light to an electrical signal, detector 18 may send the signal to monitor 14, where physiological parameters may be calculated based on the absorption of the Red and IR wavelengths in the patient's tissue 40.

In some embodiments, encoder 42 may contain information about sensor unit 12, such as what type of sensor it is (e.g., whether the sensor is intended for placement on a forehead or digit) and the wavelengths of light emitted by emitter 16. This information may be used by monitor 14 to select appropriate algorithms, lookup tables and/or calibration coefficients stored in monitor 14 for calculating the patient's physiological parameters.

Encoder 42 may contain information specific to patient 40, such as, for example, the patient's age, weight, and diagnosis. This information about a patient's characteristics may allow monitor 14 to determine, for example, patient-specific threshold ranges in which the patient's physiological parameter measurements should fall and to enable or disable additional physiological parameter algorithms. This information may also be used to select and provide coefficients for equations from which measurements may be determined based at least in part on the signal or signals received at sensor unit 12. For example, some pulse oximetry sensors rely on equations to relate an area under a portion of a PPG signal corresponding to a physiological pulse to determine blood pressure. These equations may contain coefficients that depend upon a patient's physiological characteristics as stored in encoder 42.

Encoder 42 may, for instance, be a coded resistor that stores values corresponding to the type of sensor unit 12 or the type of each sensor in the sensor array, the wavelengths of light emitted by emitter 16 on each sensor of the sensor array, and/or the patient's characteristics and treatment information. In some embodiments, encoder 42 may include a memory on which one or more of the following information may be stored for communication to monitor 14; the type of the sensor unit 12; the wavelengths of light emitted by emitter 16; the particular wavelength each sensor in the sensor array is monitoring; a signal threshold for each sensor in the sensor array; any other suitable information; physiological characteristics (e.g., gender, age, weight); or any combination thereof.

In some embodiments, signals from detector 18 and encoder 42 may be transmitted to monitor 14. In the embodiment shown, monitor 14 may include a general-purpose microprocessor 48 connected to an internal bus 50. Microprocessor 48 may be adapted to execute software, which may include an operating system and one or more applications, as part of performing the functions described herein. Also connected to bus 50 may be a read-only memory (ROM) 52, a random access memory (RAM) 54, user inputs 56, display 20, data output 84, and speaker 22.

RAM 54 and ROM 52 are illustrated by way of example, and not limitation. Any suitable computer-readable media may be used in the system for data storage. Computer-readable media are capable of storing information that can be interpreted by microprocessor 48. This information may be data or may take the form of computer-executable instructions, such as software applications, that cause the microprocessor to perform certain functions and/or computer-implemented methods. Depending on the embodiment, such computer-readable media may include computer storage media and communication media. Computer storage media may include volatile and non-volatile, removable and non-removable media implemented in any method or technology for storage of information such as computer-readable instructions, data structures, program modules or other data. Computer storage media may include, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium that can be used to store the desired information and that can be accessed by components of the system.

In the embodiment shown, a time processing unit (TPU) 58 may provide timing control signals to light drive circuitry 60, which may control when emitter 16 is illuminated and multiplexed timing for Red LED 44 and IR LED 46. TPU 58 may also control the gating-in of signals from detector 18 through amplifier 62 and switching circuit 64. These signals are sampled at the proper time, depending upon which light source is illuminated. The received signal from detector 18 may be passed through amplifier 66, low pass filter 68, and analog-to-digital converter 70. The digital data may then be stored in a queued serial module (QSM) 72 (or buffer) for later downloading to RAM 54 as QSM 72 is filled. In some embodiments, there may be multiple separate parallel paths having components equivalent to amplifier 66, filter 68, and/or A/D converter 70 for multiple light wavelengths or spectra received. Any suitable combination of components (e.g., microprocessor 48, RAM 54, analog to digital converter 70, any other suitable component shown or not shown in FIG. 2) coupled by bus 50 or otherwise coupled (e.g., via an external bus), may be referred to as “processing equipment” or “processing circuitry.”

In some embodiments, microprocessor 48 may determine the patient's physiological parameters, such as SpO₂, pulse rate, and/or respiration information, using various algorithms and/or look-up tables based on the value of the received signals and/or data corresponding to the light received by detector 18. As is described herein, microprocessor 48 may generate respiration morphology signals and determine respiration information from a PPG signal.

Signals corresponding to information about patient 40, and particularly about the intensity of light emanating from a patient's tissue over time, may be transmitted from encoder 42 to decoder 74. These signals may include, for example, encoded information relating to patient characteristics. Decoder 74 may translate these signals to enable microprocessor 48 to determine the thresholds based at least in part on algorithms or look-up tables stored in ROM 52. In some embodiments, user inputs 56 may be used to enter information, select one or more options, provide a response, input settings, any other suitable inputting function, or any combination thereof. User inputs 56 may be used to enter information about the patient, such as age, weight, height, diagnosis, medications, treatments, and so forth. In some embodiments, display 20 may exhibit a list of values, which may generally apply to the patient, such as, for example, age ranges or medication families, which the user may select using user inputs 56.

Calibration device 80, which may be powered by monitor 14 via a communicative coupling 82, a battery, or by a conventional power source such as a wall outlet, may include any suitable signal calibration device. Calibration device 80 may be communicatively coupled to monitor 14 via communicative coupling 82, and/or may communicate wirelessly (not shown). In some embodiments, calibration device 80 is completely integrated within monitor 14. In some embodiments, calibration device 80 may include a manual input device (not shown) used by an operator to manually input reference signal measurements obtained from some other source (e.g., an external invasive or non-invasive physiological measurement system).

Data output 84 may provide for communications with other devices utilizing any suitable transmission medium, including wireless (e.g., WiFi, Bluetooth, etc.), wired (e.g., USB, Ethernet, etc.), or application-specific connections. Data output 84 may receive messages to be transmitted from microprocessor 48 via bus 50. Exemplary messages to be sent in an embodiment described herein may include samples of the PPG signal to be transmitted to an external device for determining respiration information.

The optical signal attenuated by the tissue of patient 40 can be degraded by noise, among other sources. One source of noise is ambient light that reaches the light detector. Another source of noise is electromagnetic coupling from other electronic instruments. Movement of the patient also introduces noise and affects the signal. For example, the contact between the detector and the skin, or the emitter and the skin, can be temporarily disrupted when movement causes either to move away from the skin. Also, because blood is a fluid, it responds differently than the surrounding tissue to inertial effects, which may result in momentary changes in volume at the point to which the oximeter probe is attached.

Noise (e.g., from patient movement) can degrade a sensor signal relied upon by a care provider, without the care provider's awareness. This is especially true if the monitoring of the patient is remote, the motion is too small to be observed, or the care provider is watching the instrument or other parts of the patient, and not the sensor site. Processing sensor signals (e.g., PPG signals) may involve operations that reduce the amount of noise present in the signals, control the amount of noise present in the signal, or otherwise identify noise components in order to prevent them from affecting measurements of physiological parameters derived from the sensor signals.

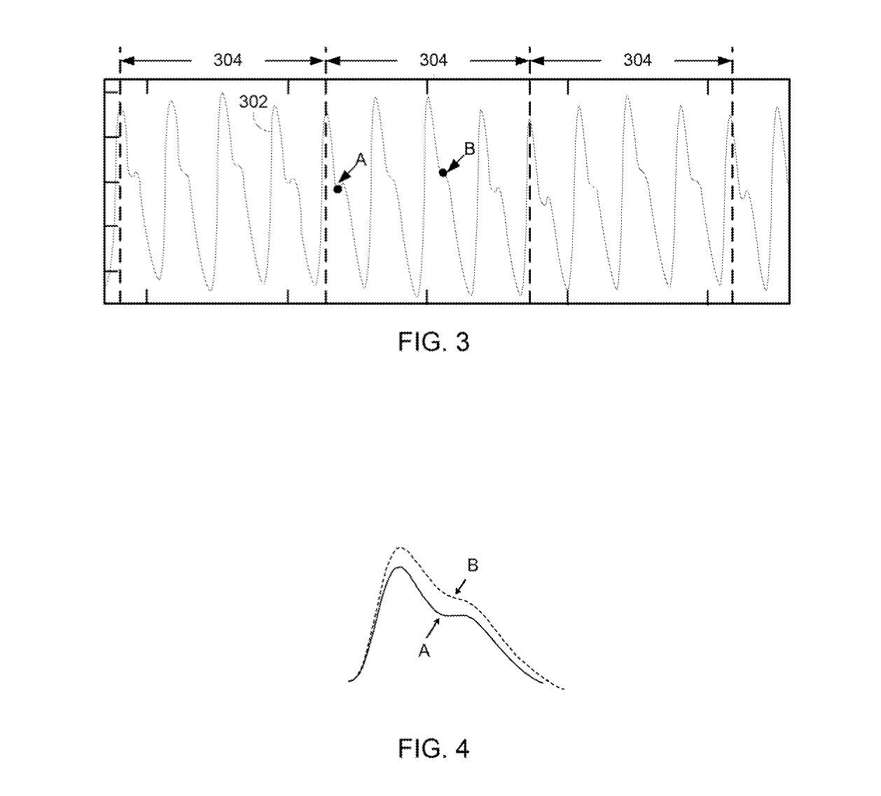

FIG. 3 shows an illustrative PPG signal 302 that is modulated by respiration in accordance with some embodiments of the present disclosure. PPG signal 302 may be a periodic signal that is indicative of changes in pulsatile blood flow. Each cycle of PPG signal 302 may generally correspond to a pulse, such that a heart rate may be determined based on PPG signal 302. Each respiratory cycle 304 may correspond to a breath. The period of a respiratory cycle may typically be longer than the period of a pulsatile cycle, such that any changes in the pulsatile blood flow due to respiration occur over a number of pulsatile cycles. The volume of the pulsatile blood flow may also vary in a periodic manner based on respiration, resulting in modulations to the pulsatile blood flow such as amplitude modulation, frequency modulation, and baseline modulation. This modulation of PPG signal 302 due to respiration may result in changes to the morphology of PPG signal 302.

FIG. 4 shows a comparison of portions of the illustrative PPG signal 302 of FIG. 3 in accordance with some embodiments of the present disclosure. The signal portions compared in FIG. 4 may demonstrate differing morphology due to respiration modulation based on the relative location of the signal portions within a respiratory cycle 304. For example, a first pulse associated with the respiratory cycle may have a relatively low amplitude (indicative of amplitude and baseline modulation) as well as an obvious distinct dichrotic notch as indicated by point A. A second pulse may have a relatively high amplitude (indicative of amplitude and baseline modulation) as well as a dichrotic notch that has been washed out as depicted by point B. Frequency modulation may be evident based on the relative period of the first pulse and second pulse. Referring again to FIG. 3, by the end of the respiratory cycle 304 the pulse features may again be similar to the morphology of A. Although the impact of respiration modulation on the morphology of a particular PPG signal 302 has been described herein, it will be understood that respiration may have varied effects on the morphology of a PPG signal other than those depicted in FIGS. 3 and 4.

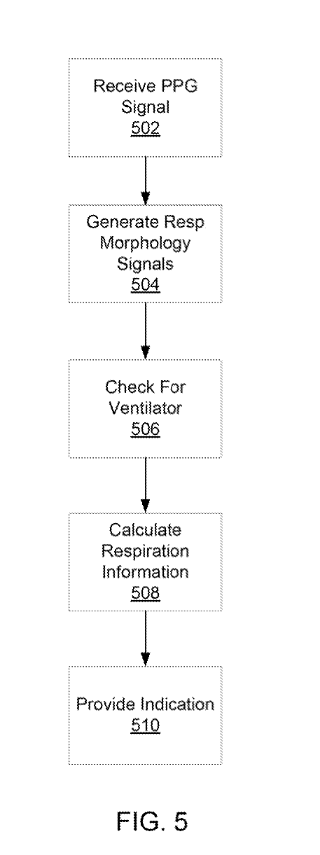

FIG. 5 shows illustrative steps for determining respiration information from a PPG signal including checking whether the patient is on a ventilator in accordance with some embodiments of the present disclosure. Although exemplary steps are described herein, it will be understood that steps may be omitted and that any suitable additional steps may be added for determining respiration information. Although the steps described herein may be performed by any suitable device, in an exemplary embodiment, the steps may be performed by monitoring system 10. At step 502, monitoring system 10 may receive a PPG signal as described herein. Although the PPG signal may be processed in any suitable manner, in an embodiment, the PPG signal may be analyzed each 5 seconds, and for each 5 second analysis window, the most recent 45 seconds of the PPG signal may be analyzed.

At step 504, monitoring system 10 may generate one or more respiration morphology signals from the PPG signal. In some embodiments, a plurality of respiration morphology signals may be generated from the PPG signal, and the plurality of respiration morphology signals may be selected as described below is step 506. In some embodiments, a particular set of respiration morphology signals may be generated from the PPG signal, for example, a down signal, a delta of second derivative (DSD) signal, and a kurtosis signal may be generated. Although respiration morphology signals may be generated in any suitable manner, in an exemplary embodiment, respiration morphology signals may be generated based on calculating a series of morphology metrics based on a PPG signal. One or more morphology metrics maybe calculated for each portion of the PPG signal (e.g., for each fiducial defined portion), a series of morphology metrics may be calculated over time, and the series of morphology metrics may be processed to generate one or more respiration morphology signals.

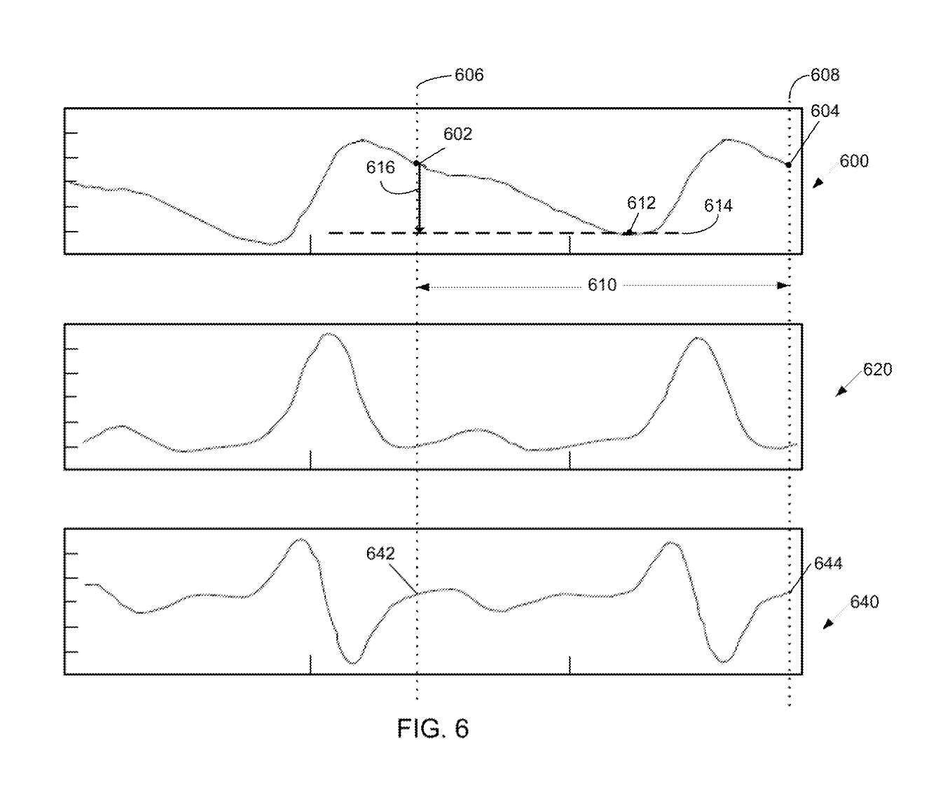

FIG. 6 depicts exemplary signals used for calculating morphology metrics from a received PPG signal. The abscissa of each plot of FIG. 6 may represent time and the ordinate of each plot may represent magnitude. PPG signal 600 may be a received PPG signal, first derivative signal 620 may be a signal representing the first derivative of the PPG signal 600, and second derivative signal 640 may be a signal representing the second derivative of the PPG signal 600. As will be described herein, morphology metrics may be calculated for portions of these signals, and a series of morphology metric calculations calculated over time may be processed to generate the respiration morphology signals. Although particular morphology metric calculations are set forth below, each of the morphology metric calculations may be modified in any suitable manner.

Although morphology metrics may be calculated based on any suitable portions of the PPG signal 600 (as well as the first derivative signal 620, second derivative signal 640, and any other suitable signals that may be generated from the PPG signal 600), in an exemplary embodiment, morphology metrics may be calculated for each fiducial-defined portion such as fiducial defined portion 610 of the PPG signal 600. Exemplary fiducial points 602 and 604 are depicted for PPG signal 600, and fiducial lines 606 and 608 demonstrate the location of fiducial points 602 and 604 relative to first derivative signal 620 and second derivative signal 640.

Although it will be understood that fiducial points may be identified in any suitable manner, in exemplary embodiments fiducial points may be identified based on features of the PPG signal 620 or any derivatives thereof (e.g., first derivative signal 620 and second derivative signal 640) such as peaks, troughs, points of maximum slope, dichrotic notch locations, pre-determined offsets, any other suitable features, or any combination thereof. Fiducial points 602 and 604 may define a fiducial-defined portion 610 of PPG signal 600. The fiducial points 602 and 604 may define starting and ending points for determining morphology metrics, and the fiducial-defined portion 610 may define a relevant portion of data for determining morphology metrics. It will be understood that other starting points, ending points, and relative portions of data may be utilized to determine morphology metrics.

An exemplary morphology metric may be a down metric. The down metric is the difference between a first (e.g., fiducial) sample of a fiducial-defined portion (e.g., fiducial defined portion 610) of the PPG signal (e.g., PPG signal 600) and a minimum sample (e.g., minimum sample 612) of the fiducial-defined portion 610 of the PPG signal 600. The down metric may also be calculated based on other points of a fiducial-defined portion. The down metric is indicative of physiological characteristics which are related to respiration, e.g., amplitude and baseline modulations of the PPG signal. In an exemplary embodiment, fiducial point 602 defines the first location for calculation of a down metric for fiducial-defined portion 610. In the exemplary embodiment, the minimum sample of fiducial-defined portion 610 is minimum point 612, and is indicated by horizontal line 614. The down metric may be calculated by subtracting the value of minimum point 612 from the value of fiducial point 602, and is depicted as down metric 616.

Another exemplary morphology metric may be a kurtosis metric for a fiducial-defined portion. Kurtosis measures the peakedness of the PPG signal 600 or a derivative thereof (e.g., first derivative signal 620 or second derivative signal 640). In an exemplary embodiment, the kurtosis metric may be based on the peakedness of the first derivative signal 620. The peakedness is sensitive to both amplitude and period (frequency) changes, and may be utilized as an input to generate respiration morphology signals that may be used to determine respiration information such as respiration rate. Kurtosis may be calculated based on the following formulae:

$$D = \frac{1}{n} {\sum }_{i = 1}^{n} {( {x}_{i}^{′} - {\overline{x}}^{′} )}^{2}$$

$$Kurtosis = \frac{1}{{nD}^{2}} {\sum }_{i = 1}^{n} {( {x}_{i}^{′} - {\overline{x}}^{′} )}^{4}$$

where:
xᵢ′=ith sample of 1^{st} derivative;
x̅′̅=mean of 1st derivative of fiducial-defined portion;
n=set of all samples in the fiducial-defined portion

Another exemplary morphology metric may be a delta of the second derivative (DSD) between consecutive fiducial-defined portions, e.g., at consecutive fiducial points. Measurement points 642 and 644 for a DSD calculation are depicted at fiducial points 602 and 604 as indicated by fiducial lines 606 and 608. The second derivative signal is indicative of the curvature of a signal. Changes in the curvature of the PPG signal 600 that can be identified with second derivative signal 640 are indicative of changes in internal pressure that occur during respiration, particularly changes near the peak of a pulse. By providing a metric of changes in curvature of the PPG signal, the DSD morphology metric may be utilized as an input to determine respiration information, such as respiration rate. The DSD metric may be calculated for each fiducial-defined portion by identifying the value of the second derivative signal 640 at the current fiducial point (e.g., fiducial point 642 of fiducial-defined portion 610) and subtracting from that the value of the second derivative signal 640 at the next fiducial point (e.g., fiducial point 644 of fiducial-defined portion 610).

Although a down metric, kurtosis metric, and DSD metric have been described, any suitable morphology metrics related to respiration may be calculated for use in generating respiration morphology signals. Other exemplary morphology metrics that may be relevant to determining a physiological parameter such as respiration information from a PPG signal may include an up metric, a skew metric, a ratio of samples metric (e.g., a b/a ratio metric or c/a ratio metric), a i b metric, a peak amplitude metric, a center of gravity metric, and an area metric. It will be understood that metrics may be determined from the original PPG signal or any derivative thereof (e.g., a down metric may be determined for each of the PPG signal, the first derivative of the PPG signal, and/or the second derivative of the PPG signal).

In some embodiments, each series of morphology metric values may be further processed in any suitable manner to generate the respiration morphology signals. Although any suitable processing operations may be performed for each series of morphology metric values, in an exemplary embodiment, each series of morphology metric values may be filtered (e.g., based on frequencies associated with respiration) and interpolated to generate the plurality of respiration morphology signals. Processing may then continue to step 506.

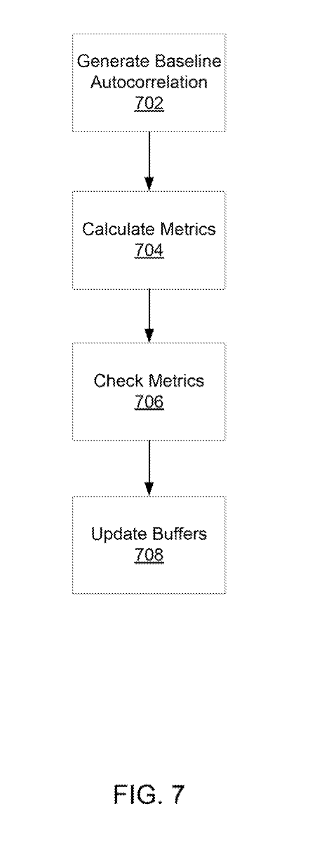

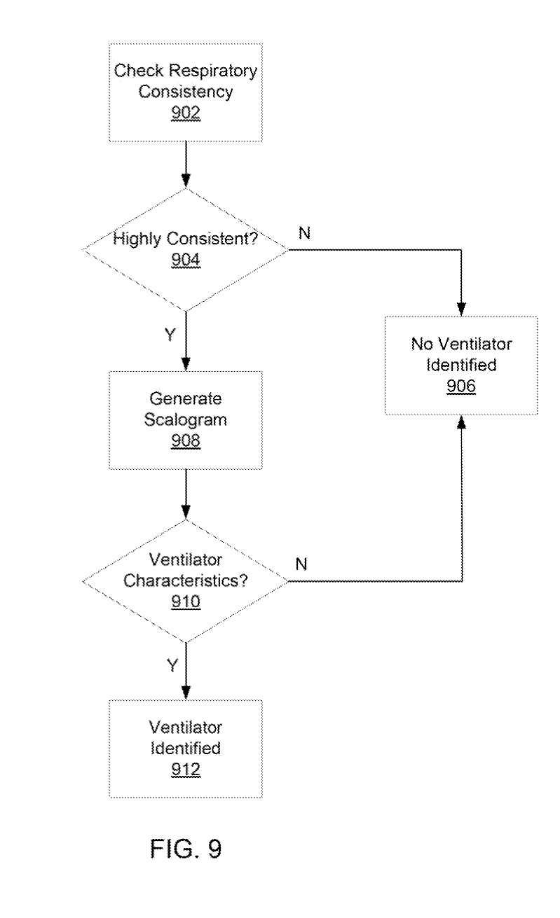

At step 506, monitoring system 10 may perform checks to see if a patient's breathing is being assisted by a ventilator. When a patient's breathing is being assisted by a ventilator, the patient's respiration is mechanically induced at a regular rate that is controlled by the ventilator. This regular pattern may result in modulations to a physiological signal (e.g., a PPG signal) that differ from the modulations that occur when a patient is breathing without assistance. In the context of a PPG signal, the PPG signal obtained from a patient breathing with the assistance of a ventilator may have strong and regular baseline modulations. As is described herein, in some embodiments, respiration morphology signals may be generated from the PPG signal, an autocorrelation signal may be generated based on the respiration morphology signals, and respiration information such as respiration rate may be calculated based on periodic information that can be identified from the autocorrelation signal. However, an autocorrelation signal generated from a ventilated patient may demonstrate strong harmonics that may make it difficult to distinguish the actual respiration rate associated with the ventilator from a respiration rate associated with the first harmonic. Moreover, the respiration rate induced by a ventilator may often be lower than a typical respiration rate of a patient, and thus the procedures for determining respiration rate may be likely to select a respiration rate associated with a harmonic typical of a higher rate rather than a respiration rate associated with the actual ventilated respiration rate. In accordance with some embodiments of the present disclosure, monitor 10 identifies when a patient's breathing is based on a ventilator (e.g., as described in accordance with FIGS. 7 and 9).

FIG. 7 shows illustrative steps for identifying a ventilated patient in accordance with some embodiments of the present disclosure. Compared to a patient that is breathing without assistance, a ventilated patient breathes in a strong and regular pattern. This pattern that is characteristic of a ventilated patient may result in a strong and regular baseline modulation to the PPG signal. In some embodiments, the baseline modulations may be analyzed to determine whether a patient's breathing is being assisted by a ventilator. Although exemplary steps for analyzing the baseline modulation of the PPG signal are described herein, it will be understood that steps may be omitted and that any suitable additional steps may be added for determining respiration information. Although the steps described herein may be performed by any suitable device, in an exemplary embodiment, the steps may be performed by monitoring system 10.

At step 702, monitor 10 may generate an autocorrelation signal based on baseline modulations to the PPG signal. Although a baseline signal may be acquired in any suitable manner, in an embodiment, a baseline signal may be acquired from the PPG signal based on sampling of the PPG signal and identifying periodic changes to the relative amplitude of the PPG signal that are not the result of amplitude modulation (i.e., that are due to the changing DC portion of the signal rather than an increase in the peak-to-peak strength of the signal). An autocorrelation may be performed on the baseline signal. The peaks of an autocorrelation correspond to portions of the signal that include the same or similar information. Thus, the peaks of the autocorrelation signal may correspond to periodic aspects of the baseline signal, the patterns of which may be indicative of a ventilated patient.

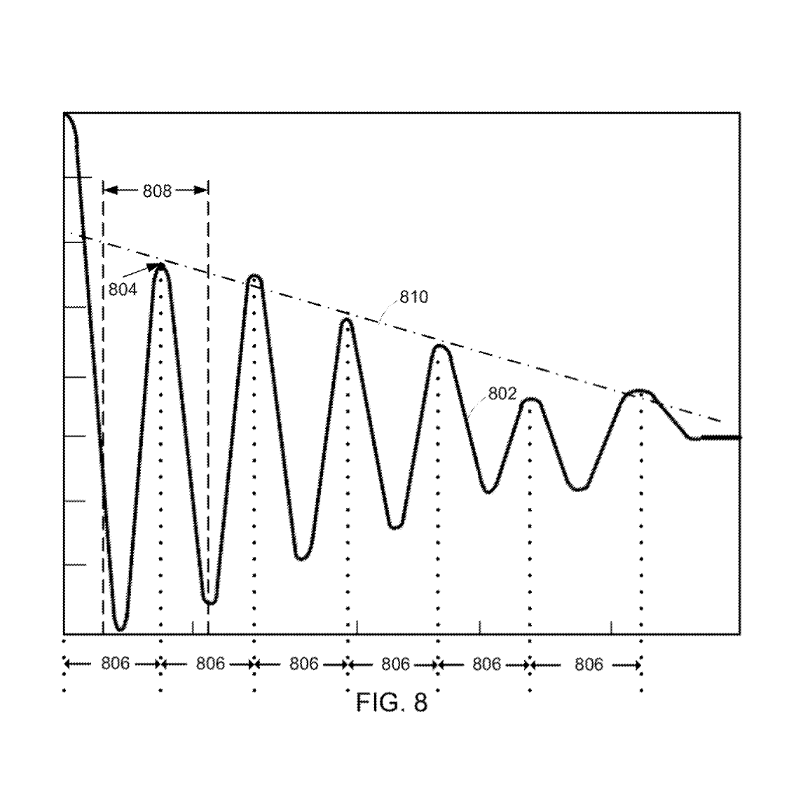

At step 704, monitor 10 may calculate one or more metrics based on the baseline autocorrelation signal. Although it will be understood that any suitable metrics may be calculated based on the baseline autocorrelation signal, in an embodiment, the metrics may be a peak amplitude metric, a time consistency metric, a frequency range metric, a slope metric, and a historical standard deviation metric. These metrics will be described in connection with FIG. 8, which shows an illustrative baseline autocorrelation sequence 802 in accordance with some embodiments of the present disclosure.

Although a peak amplitude metric may be calculated in any suitable manner, in an embodiment, the peak amplitude metric may be based on the amplitude of the first peak 804 of the baseline autocorrelation sequence 802.

Although a time consistency metric may be calculated in any suitable manner, in an embodiment, the timing 806 between each of the peaks of baseline autocorrelation sequence 802 may be determined, and the mean absolute deviation of this timing may be calculated as the time consistency metric.

Although a frequency range metric may be calculated in any suitable manner, in an embodiment, a range 808 associated with a typical respiration rate associated with ventilated breathing (e.g., 5-16 breaths per minute) may be established and it may be determined whether the first peak 804 of baseline autocorrelation sequence 802 falls within range 808.

Although a slope metric may be calculated in any suitable manner, in an embodiment, a best fit line 810 may be established for the peaks of baseline autocorrelation sequence 802. The slope metric may be the slope of the best fit line 810.

Although a historical standard deviation metric may be calculated in any suitable manner, in an embodiment, the standard deviation of the respiration rate associated the 9 previous analysis windows, or any other suitable number of previous analysis windows, may be utilized as the historical standard deviation metric.

Referring back to FIG. 7, at step 706, monitor 10 may check the metric values to determine whether the data received in the most recent 5-second data window is indicative of a patient having ventilated breathing. Although it will be understood that the metrics may be analyzed in any suitable manner, in an embodiment, all of the metrics must fall within the appropriate value range for the data window to be identified as from a patient having ventilated breathing. In some embodiments, at least a predetermined number of the metrics must fall within the appropriate value range for the data window to be identified as from a patient having ventilated breathing. In some embodiments, the data window may be identified as from a patient having ventilated breathing based on an output of a trained neural network. Although it will be understood that a trained neural network may be configured to identify ventilated breathing in any suitable manner, in an embodiment, the neural network may be trained based on training data and weights assigned to nodes associated with each of the metrics. Each node may then generate a node value associated with the assigned weight (e.g., based on the degree to which the metric calculated for the window corresponds to an ideal value) and the node values may be combined (e.g., added) and compared to a trained threshold to determine whether to identify the data window as from a patient having ventilated breathing.

At step 708, monitor 10 may update a buffer based on the result of the metric check of step 706. Because a patient with breathing assisted by a ventilator typically uses the ventilator for an extended period of time, in some embodiments, a plurality of data windows must be found to be indicative of a ventilated patient before monitor 10 determines that the patient's breathing is being assisted by a ventilator. Although it will be understood that any number of a plurality of data windows may be analyzed in any suitable manner, in some embodiments, two data buffers may be populated and analyzed. Although the data buffers may be implemented in any suitable manner, in some embodiments, monitor 10 may include a short-term data buffer and a long term data buffer.

Although a short-term data buffer may be implemented in any suitable manner, in some embodiments, the short-term data buffer may be updated for each 5-second data window, and may include 9 data elements (e.g., for the 9 most recent 5-second data windows) that update on a first in first out basis. Whether the patient's breathing is being assisted by a ventilator may be determined from the short term buffer. Although this may be determined in any suitable manner, in an embodiment, if 8 of the 9 data elements of the short-term buffer indicate a ventilated patient, monitor 10 may determine that the patient's breathing is being assisted by a ventilator.

Although a long-term data buffer may be implemented in any suitable manner, in some embodiments, the long-term data buffer may be updated for each 5-second data window based on the determination of a ventilated patient from the short-term data buffer (e.g., each data element of the long-term buffer may be based on the entirety of the short-term buffer for a particular 5-second data window). The long-term data buffer may be continually updated on a first in first out basis.

In some embodiments, the long term value may also indicate a ventilated patient based on one or more additional factors, such as a ventilation age value or a ventilation latch value. During an initial onset of ventilation, the characteristics of the ventilation may not be as regular as they might be after ventilation has been ongoing during an extended period. In some embodiments, if it is during an initial onset of ventilation (e.g., if recent data did not indicate a ventilated patient), then once a sufficient number of the data elements of the long-term buffer indicate a ventilated patient it may be desirable to continue to indicate that the patient is ventilated even if the short-term buffer temporarily does not indicate a ventilated patient. In an embodiment, a ventilation age value may indicate the elapsed time since the output of the short-term buffer began to indicate a ventilated patient (e.g., the elapsed time since 8 of the 9 data elements of the short term buffer indicated a ventilated patient). In an embodiment, a ventilation latch may be set once a sufficient number of the data elements of the long-term buffer (e.g., 7 of 9) indicate a ventilate patient. In an embodiment, the data element of the long-term buffer associated with the most recent 5-second data window may indicate a ventilated patient if the ventilation age is less than a threshold (e.g., 200 seconds) and the ventilation latch is set (i.e., even if the current short-term buffer would not otherwise indicate a ventilated patient). Once the long-term buffer is updated, processing may continue to step 508 of FIG. 5.

Before returning to step 508, FIG. 9 also shows illustrative steps for identifying a ventilated patient (i.e., at step 506) in accordance with some embodiments of the present disclosure as an alternative to the process shown in FIG. 8 or in addition to the process shown in FIG. 8. At step 902, monitor 10 may calculate a consistency value based on recently determined respiration rate vales. Although it will be understood that a consistency value may be calculated in any suitable manner, in some embodiments, a consistency value may be based on a standard deviation of recent respiration rate values, a proportion of respiration rate values falling within a particular range, any other suitable procedure for testing data consistency, or any combination thereof. Processing may then continue to step 904.

At step 904, monitor 10 may determine whether the respiration rate is highly consistent, which in an embodiment, may provide an indication that it is likely that the patient's breathing is being assisted by a ventilator. Although it will be understood that the consistency of the respiration rate data may be evaluated in any suitable manner, in an embodiment, the consistency value may be compared to a predetermined consistency threshold. For example, the reported respiration rate may be determined consistent if it is within 1 breath per minute over a specified period of time, for example 5 minutes. If the consistency value fails to meet the consistency threshold (e.g., if the respiration rate values are not determined to be highly consistent), processing may continue to step 906. If the consistency value meets the consistency threshold (e.g., if the respiration rate values are determined to be highly consistent), processing may continue to step 908.

At step 906, monitor 10 determines that the patient has not been identified as having ventilated breathing. Processing may then return to step 508 of FIG. 5.

At step 908, monitor 10 may perform a wavelet transform, such as a continuous wavelet transform, on the PPG signal and generate a scalogram. In the discussion that follows, a “scalogram” may be understood to include all suitable forms of rescaling including, but not limited to, the original unscaled wavelet representation, linear rescaling, any power of the modulus of the wavelet transform, or any other suitable resealing. In addition, for purposes of clarity and conciseness, the term “scalogram” shall be taken to mean the wavelet transform, T(a,b) itself, or any part thereof. For example, the real part of the wavelet transform, the imaginary part of the wavelet transform, the phase of the wavelet transform, any other suitable part of the wavelet transform, or any combination thereof is intended to be conveyed by the term “scalogram.” It will be understood that the term scalogram may refer to any suitable scalogram or modification thereof, e.g., a combined sum scalogram or sum scalogram vector as described herein.

Monitor 10 may perform a wavelet transform such as a continuous wavelet transform. The continuous wavelet transform of a signal x(t) in accordance with the present disclosure may be defined as:

$$T ( a , b ) = \frac{1}{\sqrt{a}} {\int }_{- ∞}^{∞} x ( t ) ψ * ( \frac{t - b}{a} ) d t$$

where:
a=scale value;
b=shift parameter; and
ψ(t)=wavelet function and * denotes complex conjugate.

The resulting wavelet may be summed to generate a sum scalogram corresponding to the scales of the wavelet transform. Processing may then continue to step 910.

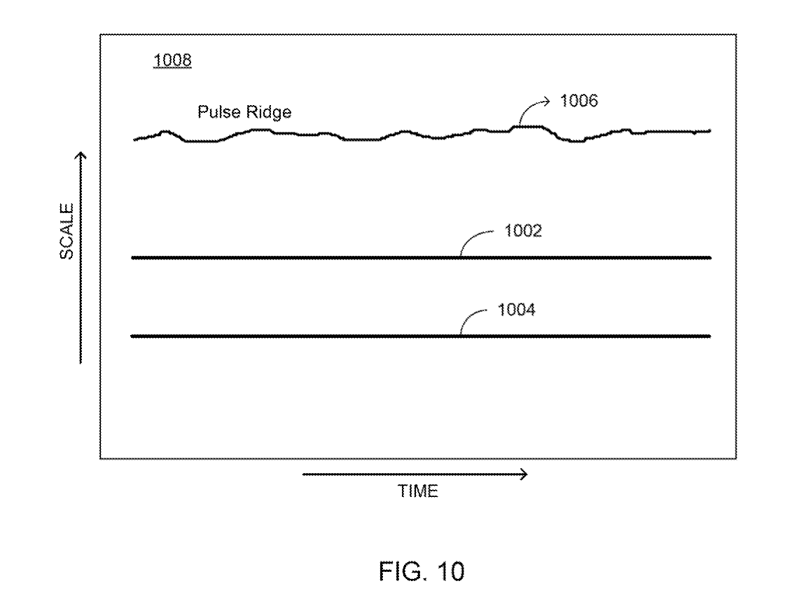

At step 910, monitor 10 may determine whether the sum scalogram includes characteristics that are indicative of ventilated breathing. For example, it has been observed that when a patient is ventilated, at least three distinct bands (i.e., bands of energy across scales) appear in a scalogram generated from a wavelet transform of the patient's PPG signal. These include a pulse band, a breathing band at the ventilated respiration rate and a band at a second respiration rate located at a multiple (e.g., twice) the actual rate. This is illustrated in FIG. 10, which shows a simplified scalogram 1008, pulse band ridge 1006, and breathing band ridges 1002 and 1004. Scalogram 1008 represents a scalogram generated using a continuous wavelet transform of a PPG signal from a patient receiving positive airway pressure ventilation. Ridge 1002 is located at a scale about twice that of ridge 1004. Many conventional respiration rate algorithms would identify the scale of ridge 1002 as the actual respiration rate because of the presence of higher frequency components when a patient is being ventilated. It is therefore desirable to identify when a patient is being ventilated in order to avoid reporting the higher frequency erroneous respiration rate instead of the true lower frequency rate.

In determining whether respiration is ventilated, monitor 10 may analyze ridges 1002 and 1004 as well as their associated bands (not shown) to determine their respective consistency. When ridges 1002 and 1004, their associated respective bands, or both are very consistent, this is indicative of ventilated respiration. Consistency may be determined using any suitable technique, such as comparing a standard deviation, or average standard deviation, of the measured rate against any suitable predetermined or dynamically determined thresholds. Consistency may manifest itself as the size of the scale range of a respective band or ridge (e.g., a substantially straight horizontal line ridge is very consistent). While both bands may be used in determining whether a patient is being ventilated, when it is determined that the patient is, indeed, being ventilated, the band corresponding to the lower frequency is selected as corresponding to the respiration rate.

If the scalogram is identified as corresponding to a ventilated patient, processing may continue to step 912. At step 912, monitor 10 may determine that the patient has been identified as having ventilated breathing. In some embodiments, monitor 10 may set any appropriate flags or indicators for use in additional processing and/or may identify the respiration rate that corresponds to ventilated breathing. Processing may then return to step 508 of FIG. 5.

If the scalogram is not identified as corresponding to a ventilated patient, processing may continue to step 906. At step 906, monitor 10 may determine that the patient has not been identified as having ventilated breathing. Processing may then return to step 508 of FIG. 5.

Referring again to FIG. 5, at step 508, monitor 10 may calculate respiration information such as respiration rate. Although respiration information such as respiration rate maybe calculated in any suitable manner, in some embodiments, the calculation may be based at least in part on whether the patient breathing was determined to be assisted by a ventilator in step 506. In some embodiments, if the patient breathing was determined to be assisted by a ventilator, the respiration rate may be based on the band of the scalogram that was determined to be associated with ventilated breathing. In some embodiments, if the patient breathing was determined to be assisted by a ventilator, the respiration rate may be calculated in any other suitable manner (e.g., based on an analysis of a wavelet transform, based on an autocorrelation of respiration morphology signals, etc.). Processing may then continue to step 510.

At step 510, monitor 10 may provide an indication of ventilated breathing based on the determination at step 506. Although it will be understood that monitor 10 may provide an indication of a held breath event in any suitable manner, in an embodiment, monitor 10 may post only the respiration rate value associated with a ventilator, stop posting a respiration rate value, provide a visual indication, provide an audible indication, provide a transmitted indication, provide any other suitable indication or response, or any combination thereof.

Although it will be understood that monitor 10 may stop posting respiration rate values in any suitable manner (e.g., immediately upon the identification of a ventilated patient), in some embodiments, monitor 10 may stop posting respiration rate based on how long the held breath event has persisted. In some embodiments, if a patient has been ventilated for longer than a threshold duration, monitor 10 may cease posting of respiration rate values. In some embodiments, monitor 10 may have a number of criteria under which the respiration rate may not be posted (e.g., a weak respiration signal, patient speech interfering with the measurement of respiration, etc.). An indication of a ventilated patient event may be combined with these other criteria, such that if the total duration of all of the events exceeds a threshold, a respiration rate value may not be posted by monitor 10.

It may also be desired to provide an indication of a ventilated patient, such as a visual indication, audible indication, or transmitted indication. Although it will be understood that a visual indication may be provided in any suitable manner, in some embodiments, a visual indication may be provided on display 20 as an icon, text, intermittent flashing, changes to display color, any other suitable visual indication of an indication, or any combination thereof.

Although it will be understood that an audible indication may be provided in any suitable manner, in some embodiments, an audible indication may be provided by speaker 22 as a spoken message, indication sound, any other suitable audible indication of an indication, or any combination thereof.

Although it will be understood that a transmitted indication message may be provided in any suitable manner, in some embodiments, a transmitted indication message may be provided by data output 84 to any suitable receiving device such as a central nurse station, smart phone, computing unit, medical pager, medical database, any other suitable receiving device, or any combination thereof.

In some embodiments, detection of ventilated respiration may be used to modify the processing of signals from which respiration information, such as respiration rate is, is derived. For example, because ventilated respiration provides a consistent respiration rate, less filtering may be needed when it is known that a patient is being ventilated. In some embodiments, upon detection of ventilated respiration, monitor 10 may reduce (e.g., halve) the cut-off frequency of one or more high pass filters that act upon the input physiological signal from which respiration information is determined. The amount of filtering reduction may, in some embodiments, be dynamic and based on, for example, a confidence level associated with whether the patient is being ventilated. In some embodiments, detection of ventilated breathing may be used to determine to increase the amount of filtering when a very consistent respiration is to be expected.

The foregoing is merely illustrative of the principles of this disclosure and various modifications may be made by those skilled in the art without departing from the scope of this disclosure. The above described embodiments are presented for purposes of illustration and not of limitation. The present disclosure also can take many forms other than those explicitly described herein. Accordingly, it is emphasized that this disclosure is not limited to the explicitly disclosed methods, systems, and apparatuses, but is intended to include variations to and modifications thereof, which are within the spirit of the following claims.

What is claimed is:

1. A computer-implemented method comprising:
receiving a photoplethysmograph (PPG) signal;
generating, using processing circuitry, at least one signal indicative of respiration consistency based on the PPG signal;
identifying, using the processing circuitry, a presence of ventilated breathing based on the at least one signal indicative of respiration consistency; and
providing, using the processing circuitry, an indicator of ventilated breathing based on the identifying of the presence of ventilated breathing.

2. The method of claim 1, wherein generating at least one signal indicative of respiration consistency comprises:
performing a wavelet transform of the PPG signal;
generating a scalogram based on the wavelet transform;
identifying a first portion of the scalogram and a second portion of the scalogram associated with respiration; and
determining the consistency of the first portion and of the second portion.

3. The method of claim 2, wherein the first portion comprises a first scale band and the second portion comprises a second scale band distinct from the first scale band and associated with characteristic frequencies lower than those of the first scale band, the method further comprising calculating a respiration rate based on characteristic frequencies associated with the second scale band when it is determined that ventilated breathing is present.

4. The method of claim 1, wherein generating at least one signal indicative of respiration consistency comprises:
extracting a baseline signal from the PPG signal;
performing an autocorrelation of the baseline signal to generate an autocorrelation signal; and
calculating at least one metric based on the autocorrelation signal.

5. The method of claim 4, wherein the at least one metric comprises at least one of a peak amplitude metric, a time consistency metric, a peak within frequency range metric, a normalized slope metric, and a historical standard deviation metric.

6. The method of claim 1, wherein the generating, identifying and providing steps are performed for consecutive time windows of the PPG signal.

7. The method of claim 6, further comprising:
determining whether at least a threshold number of time windows in a predetermined number of the consecutive time windows are associated with ventilated breathing; and
determining a presence of ventilatory breathing across the predetermined number of consecutive time windows.

8. The method of claim 1 further comprising determining respiration information based on the presence of ventilated breathing.

9. The method of claim 8 wherein the respiration information comprises respiration rate.

10. A system comprising:
an input that receives a photoplethysmograph (PPG) signal from a sensor; and
processing circuitry for:
generating at least one signal indicative of respiration consistency based on the PPG signal,
identifying a presence of ventilated breathing based on the at least one signal indicative of respiration consistency, and
providing an indicator of ventilated breathing based on the identifying of the presence of ventilated breathing.

11. The system of claim 10, wherein the processing circuitry is further configured for:
performing a wavelet transform of the PPG signal;
generating a scalogram based on the wavelet transform;
identifying a first portion of the scalogram and a second portion of the scalogram associated with respiration; and
determining the consistency of the first portion and of the second portion.

12. The system of claim 11, wherein the first portion comprises a first scale band and the second portion comprises a second scale band distinct from the first scale band and associated with characteristic frequencies lower than those of the first scale band, the processing circuitry further configured for calculating a respiration rate based on characteristic frequencies associated with the second scale band when it is determined that ventilated breathing is present.

13. The system of claim 10, wherein the processing circuitry is further configured for:
extracting a baseline signal from the PPG signal;
performing an autocorrelation of the baseline signal to generate an autocorrelation signal; and
generating at least one metric based on the autocorrelation signal.

14. The system of claim 13, wherein the at least one metric comprises at least one of a peak amplitude metric, a time consistency metric, a peak within frequency range metric, a normalized slope metric, and a historical standard deviation metric.

15. The system of claim 10, wherein the processing circuitry is configured to perform the generating, identifying and providing steps for consecutive time windows of the PPG signal.

16. The system of claim 15, wherein the processing circuitry is further configured for:
determining whether at least a threshold number of time windows in a predetermined number of the consecutive time windows are associated with ventilated breathing; and
determining a presence of ventilatory breathing across the predetermined number of consecutive time windows.

17. The system of claim 10, wherein the processing circuitry is further configured for determining respiration information based on the presence of ventilated breathing.

18. The system of claim 17 wherein the respiration information comprises respiration rate.

19. A non-transitory computer-readable medium having computer program instruction stored thereon for performing the method comprising:
receiving a photoplethysmograph (PPG) signal;
generating at least one signal indicative of respiration consistency based on the PPG signal;
identifying a presence of ventilated breathing based on the at least one signal indicative of respiration consistency; and
providing an indicator of ventilated breathing based on the identifying of the presence of ventilated breathing.

20. The computer-readable medium of claim 19, wherein generating at least one signal indicative of respiration consistency comprises:
performing a wavelet transform of the PPG signal;
generating a scalogram based on the wavelet transform;
identifying a first portion of the scalogram and a second portion of the scalogram associated with respiration; and
determining the consistency of the first portion and of the second portion.
